(19) United States
(12) Patent Application Publication
(10) Pub. No.: US 2002/0146834 A1
(43) Pub. Date: Oct. 10, 2002

(54) Device and method for in vitro detection of blood

(76) Inventors: Gavriel Meron, Petach Tikva (IL); Itamar Willner, Mevasseret-Zion (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 09/995,797

(22) Filed: Nov. 29, 2001

Related U.S. Application Data
(63) Continuation of application No. 09/525,016, filed on Mar. 14, 2000, now abandoned.

(51) Int. Cl. G01N 33/72

(52) U.S. Cl. 436/66; 422/57

(57) ABSTRACT

The present invention relates to a device and system for the in vitro detection of blood.

The device comprises a support having immobilized thereon at least one poly electrolyte reactant, such as poly acrylic acid, capable of reacting with blood, said reaction resulting in an optical change.

The system comprises the device according to the invention in which the support is in communication with a detecting unit, capable of detecting a reaction resulting in an optical change between the poly electrolyte reactant and blood.

The support and immobilized reactant are contacted in vitro with a sample which possibly contains blood. The reaction occurring between the reactant and blood is detected, either while the substrate is in the sample or when it is retrieved from the sample, either by eye or by the detecting unit.

### FIELD OF THE INVENTION

The present invention relates to a device and method for the in Vitro detection of the presence and/or concentration of blood or components of blood. The present invention also teaches the application of the device as a blood detection kit.

### BACKGROUND OF THE INVENTION

Medical detection kits on the market today test for substances such as urine and blood glucose, cholesterol and blood urea nitrogen. Most kits are dry chemistry kits and are available as thin strips, which may be either film-coated or impregnated. The coatings on the strip may be introduced as single or multiple layers. Most strips consist of a paper or plastic base containing reactive chemical components. The latter almost always consists of a multiplicity of chemicals, indicators, and biologically active agents such as highly purified enzymes, with which an analyte may react.

These detection kits can be used by patients in their homes and doctors in their offices.

The main advantages of dry chemistry kits over wet chemical procedures are their greater consistency and reliability, as well as their longer shelf life.

Currently, there are several ways to test for the presence of blood in body fluids and waste. Almost all fecal occult blood (FOB) tests in use today are based on the peroxidase property of hemoglobin as discussed, for example, in U.S. Pat. Nos. 5,490,969, 5,081,040, and 4,017,261. Kits for the detection of FOB using this chemistry are described in U.S. Pat. Nos. 5,447,868 and 5,563,071.

The usual technique used to screen for hematuria—blood in the urine—is the dipstick method which also uses the peroxidase property of hemoglobin. Erythrocytes (red blood cells) hemolyze on the reagent strip liberating free hemoglobin. Orthotolidine is impregnated on the strip and the free hemoglobin catalyzes its oxidation, producing a blue color. The intensity of the color change is proportional to the amount of blood in the urine.

A drawback of the above methods is that since the chemical detection of hemoglobin is based on its peroxidase activity, peroxidase activity found in many meats and vegetables often interferes with such tests. Strict dietary restrictions are thus required prior to a chemical hemoglobin test.

An immunological test for hemoglobin, and therefore blood as well, which makes use of hemoglobin antibodies is described in U.S. Pat. No. 4,920,045. A kit for detection of hemoglobin A1c, using a one-step immunoassay method based on anti-hemoglobin antibodies, is discussed in U.S. Pat. No. 5,932,480.

A general drawback of most current systems for detecting blood is that they require relatively expensive reagents.

### SUMMARY OF THE INVENTION

The present invention teaches a device, system and method for the in vitro detection of the presence and/or concentration of blood or a component of blood. The invention is based on cheap, readily available materials and does not suffer the drawbacks of blood tests in use today.

A blood component that can be detected using this invention is, i.e. hemoglobin, The device, system and method of the invention can be readily applied in medical diagnostic kits.

It will be appreciated that the term “blood” in the present invention refers to blood or any one of its components or to a combination of its components.

More specifically, the present invention provides a device for the in vitro detection of blood comprising a support having immobilized thereon at least one poly electrolyte reactant capable of reacting with blood, said reaction resulting in an optical change.

The support may be, for example, glass or plastic or any support capable of immobilizing thereon a poly electrolyte reactant. It will be appreciated that the support may be a solid support or a media support, wherein the media support may constitute a liquid phase, for example a suspension.

The poly electrolyte reactant may be for example poly acrylic acid (PAA), poly aspartic acid, poly glutamic acid or cellulose acetic acid, and is capable of being immobilized onto the support and of reacting with blood whereas the reaction results in an optical change on the support.

The present invention further provides a system for determining in vitro the presence and/or the concentration of blood comprising a support having immobilized thereon at least one poly electrolyte reactant capable of reacting with blood, said reaction resulting in an optical change and a detecting unit, in communication with the support, capable of detecting a reaction resulting in an optical change between the poly electrolyte reactant and blood.

The support and immobilized reactant are contacted in vitro with a sample which possibly contains blood. The reaction occurring between the reactant and blood is detected, either while the substrate is in the sample or when it is retrieved from the sample, either by eye or by the detecting unit.

Thus, the present invention also provides a method for determining the presence and/or concentration of blood in a sample. The method comprises the steps of 1) introducing the sample to a support, the support having immobilized thereon at least one reactant capable of reacting with blood, said reaction resulting in an optical change; and 2) receiving optical data from the support. The method may further comprise the step of analyzing the optical data received from the support.

The present invention further provides a diagnostic kit for the in vitro detection of blood. The kit may comprise the device or the system according to the invention.

### BRIEF DESCRIPTION OF THE DRAWINGS

The present invention will be understood and appreciated more fully from the following detailed description taken in conjunction with the appended drawings in which.

FIGS. 1A and 1B are schematic side and top views of the device according to the invention;

FIG. 1C is a schematic top view of the device according to the invention after a reaction between the reactant and blood has occurred;

FIG. 2 is a schematic illustration of the system according to the invention

FIGS. 3A-E show the spectra obtained for blood solutions at a concentration of 2.5 mg/ml.

FIGS. 3B-3E show the absorption spectra of the support in a blood solution obtained at 90 seconds, 120 seconds, 150 seconds and 180 seconds respectively; and

FIGS. 4A-F show the spectra obtained for blood solutions at a concentration of 8 mg/ml.

FIGS. 4B-4F show the spectra obtained at 10 seconds, 30 seconds, 45 seconds, 60 seconds and 90 seconds respectively.

### DETAILED DESCRIPTION OF THE INVENTION

The device and system of the invention can be used for the detection of the presence and/or concentration of blood in a sample. Thus, the device and/or system can be utilized in various brood detection kits.

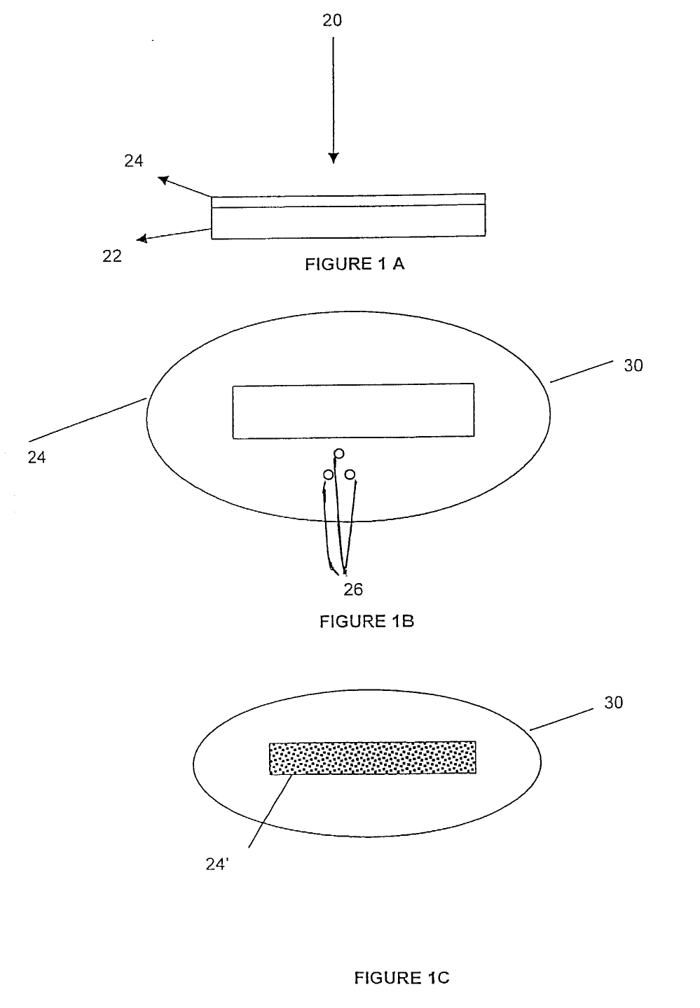

Reference is made to FIGS. 1A and 1B in which the device 20 of the invention is schematically represented. The device 20, meant for the in vitro detection of blood or a component of blood in a sample, comprises a support 22 onto which a poly electrolyte reactant layer 24 is immobilized. The device is introduced into sample 30 such that any blood 26 present in sample 30 is brought into contact with the reactant layer 24.

The sample 30 may contain human or animal body fluids or any medium for which it is desired to determine the presence or concentration of blood. The device 20 can be used for the detection of blood as a diagnostic instrument or as an indicator of the purity of the medium etc.

The support 22 can be made of a silica, such as glass, or plastic such as nylon or other plastics capable of immobilizing thereon the reactant layer 24. The support may be a media support which constitutes a liquid phase such as a suspension (not shown in the figures).

Reactant layer 24 is a layer of a poly electrolyte, such as poly acrylic acid (PAA), poly aspartic acid, poly glutamic acid or cellulose acetic acid, or a combination thereof, which is capable of being immobilized onto the support 22 and is capable of reacting with blood 26 whereas the reaction results in an optical change detectable by eye or by a detecting unit. The immobilization of the reactant to the support depends on the specific characteristics of both reactant and support. Poly electrolytes may be applied directly to the support in which case the forces involved in the immobilization of the poly electrolyte reactant to the support are electrostatic interactions, hydrogen bonding or hydrophilic interactions. The immobilization of the reactant to the support will be further demonstrated in reference to the examples and experiments below.

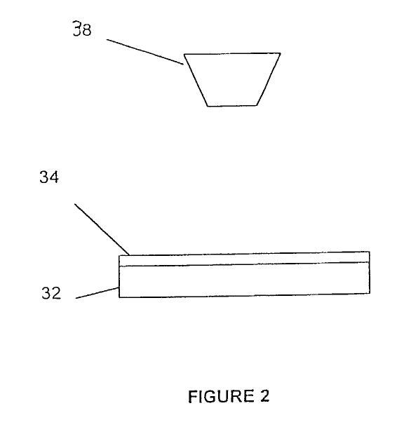

When the reactant layer 24 comes in contact with blood 26, a reaction or interaction occurs between the charged polymer reactant and components of the blood, resulting in the deposition or binding of the blood to the reactant layer 24, accompanied by an optically detectable change to the support (shown as 24′ in FIG. 1C). This change could include a change in optical density, in colors in reflectance, etc The reaction can be detected visually by the human eye or can be detected by other suitable detecting means, Reference is now made to FIG. 2 which is a schematic illustration of the system of the invention. The system comprises a support 32, to which a poly electrolyte reactant layer 34 is immobilized, and a detecting unit 38 that is in communication with the support 32. The detecting unit 38 may be any unit capable of optically detecting and reporting the optical change brought about by the reaction of the reactant layer 34 with blood. Any suitable optical mechanical detecting unit such as a spectrophotometer or reflectance meter or any suitable imaging device may be used.

Preferably, the reactant layer should be homogeneous and substantially non-absorbing in the wavelength region being used for detection, prior to the reaction. Also the support itself also should be substantially non-absorbing in this region.

It should be readily apparent that the nature and magnitude of the change in optical density, color, or other property of the support having the reactant immobilized thereon, is dependent on the concentration of the blood in the sample. In some cases, the concentration of the blood or blood component may need to be amplified. Concentration enhancement procedures are well known to those skilled in the art. Pre-processing to rid the sample of residue and contaminants may also be necessary. Again these procedures are well known to those skilled in the art.

If reflectance methods are used to track changes in the system of the invention, either a reflective layer or reflective materials can be added to the system.

The device and system of the invention may be used in medical diagnostic kits for determining the presence of blood or hemoglobin in body fluids. Quantitative results may also be obtained. For example, a plastic support, such as a poly urithane (isoplast) substrate, coated with a poly electrolyte such as PAA could be used to produce a diagnostc stick, strip or plate. The isoplast support having PAA immobilized onto it could be introduced to a body fluid sample, such as a urine or gastric fluids sample and if blood is present in the sample it will be deposited by the PAA layer and cause a change of color of the support, thus indicating the presence of blood in the sample. The deposit can be detected by eye. Quantitative indication of the amount of hemoglobin in the sample can be obtained by monitoring hemoglobin's blue absorbance at 412 nm, using a spectrophotometer.

A method for detecting the presence and/or concentration of blood in a sample is provided in which a support having a poly electrolyte immobilized thereon is contacted with a sample suspected of containing blood. Due to the electrochemical nature of the reaction between the reactant (poly electrolyte) and blood, results are usually immediately obtained. The substrate is then viewed, either while in the sample or after being retrieved from the sample, and it is determined, either by eye or by using a suitable detecting unit, whether an optical change has occurred. The presence of blood in the sample can thus be detected and/or the concentration of the blood present in the sample may be further determined by comparison of the results detected in the sample to a pre calibrated system with known concentrations of blood, For the determination of the concentration of blood in the sample the detecting unit may be in communication with any suitable analyzing unit for performing the necessary comparisons and calculations.

The invention will be further described and illustrated by the following example and figures.

### EXAMPLE

A support made of a plastic such as the poly urthane Isoplast® is coated with poly electrolytes such as poly aspartic acid, poly glutamic acid, cellulose acetic acid, poly acrylic acid (PM) or a combination thereof. The poly electrolytes are immobilized onto the support through electrostatic interactions, hydrogen bonding and/or hydrophilic interactions. The poly electrolyte layer induces the deposition of blood through an interaction between the charged poly electrolyte and blood components.

### Experiment

Supports made of Isoplast® plates (4 cm×4 cm) were cleaned with a detergent solution, rinsed with a large amount of ultrapure water and dried. A 20% (w/w) aqueous solution of polyacrylic acid (PAA) was prepared. The PAA (Aldrich Chemical Co.) had a MW of 250,000. 0.7-0.8 ml of the aqueous PAA solution was spread onto a dry, clean Isoplast® plate. After evaporating the water, the coating's weight was approximately 0.01 gr (5-6 mg/cm²).

Blood samples were diluted with phosphate buffer solution (PBS) in a volumetric flask to obtain blood solutions of different concentrations ranging from 2.5 to 25 mg/ml. The phosphate buffer solution (PBS) was prepared from 1.345 g Na₂HPO₄, 0.125 gr NaH₂PO₄ and 5.171 g KCl (Merck) in 500 ml water, with the pH adjusted to 7.2. All the blood samples used in these experiments were taken from the same donor and fresh samples were prepared immediately prior to each measurement.

Spectral measurements of the Isoplast® plates, before and after deposition of blood, were made using a UVICON-860 spectrophotometer. The plates were scanned in the 380 to 430 nm region.

The process of blood coagulation on the polymer coated Isoplast® plates was observed visually, by eye, as the formation of a brown-reddish precipitant The process was also monitored by using a spectrophotometer. The spectrum of the PAA coated Isoplast® plates in the region of 380-430 nm was recorded before each deposition of blood. The PAA coated Isoplast® surface was exposed to 1 ml of fresh blood solution at a given concentration. The blood was removed every 15 seconds and replaced with a fresh sample. The spectra obtained for the plate after exposure to blood were compared with the spectra of blood solutions having concentrations ranging from 0.5 to 2.5 mg/ml.

### Results

Solutions with blood concentrations of 10, 8, 7, 3.5 and 2.5 mg/ml were tested. All solutions with concentrations in excess of 2.5 mg/ml showed aggregation and precipitation of blood after 10-30 seconds of exposure. The exact amount of time needed to observe precipitation was a function of the concentration of the solution. In the experiments with blood solutions of 2.5 mg/ml concentration, the change in plate transparency was observed only after 60 seconds. After 60-90 seconds, an adsorbtion of different sized particles was observed. These particles did not disappear after washing the plate with water or an HCl solution.

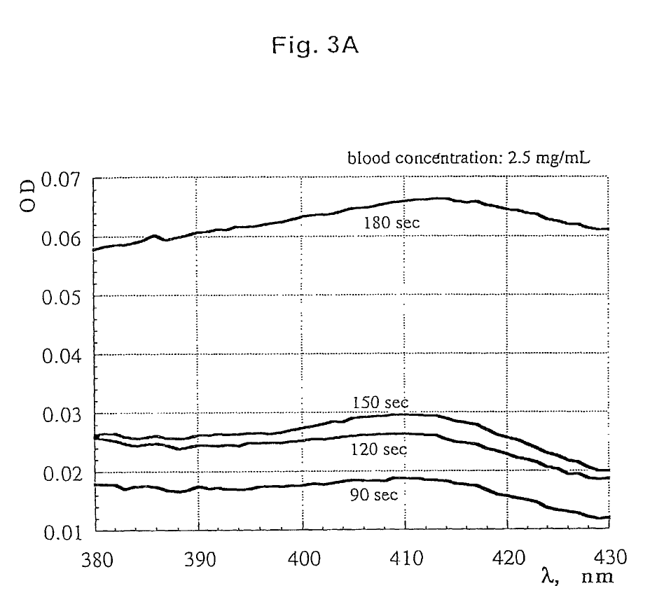

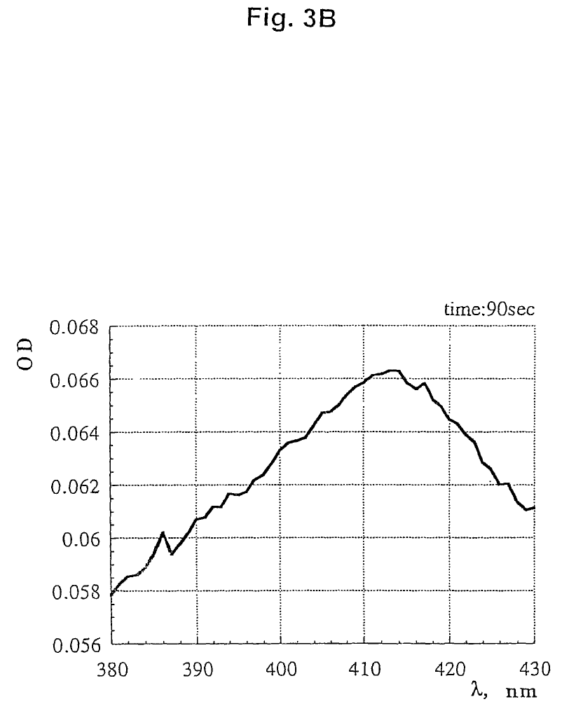

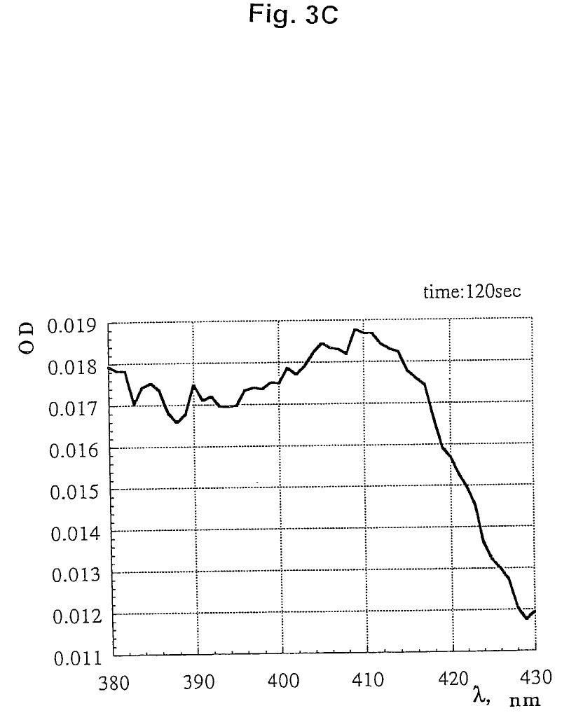

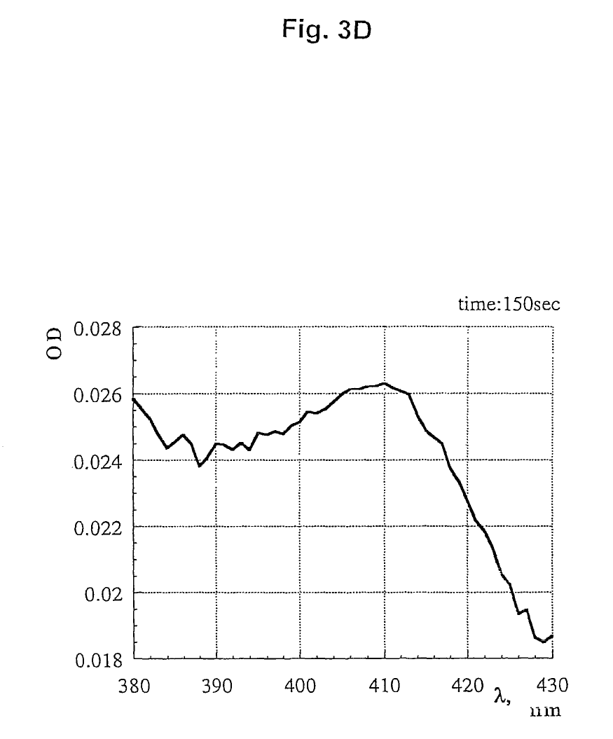

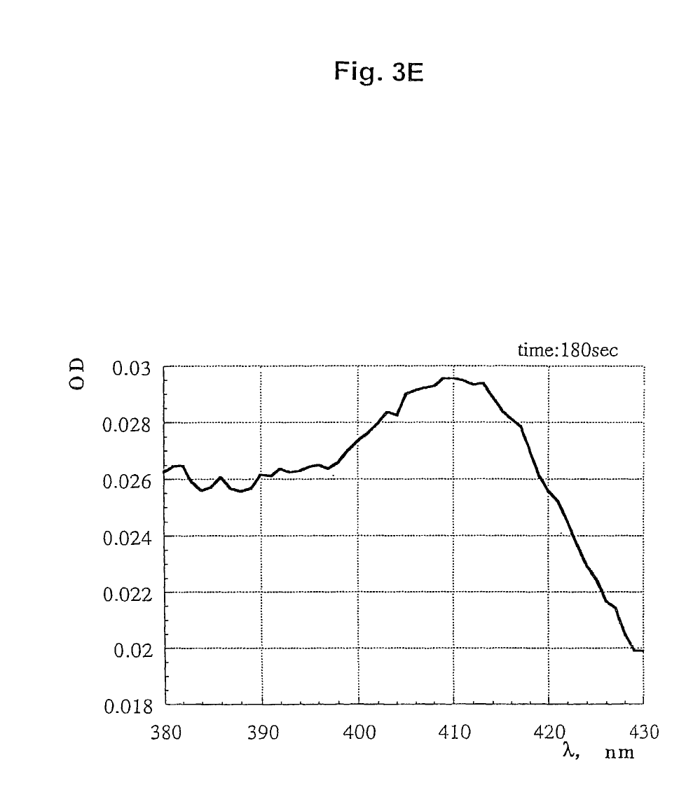

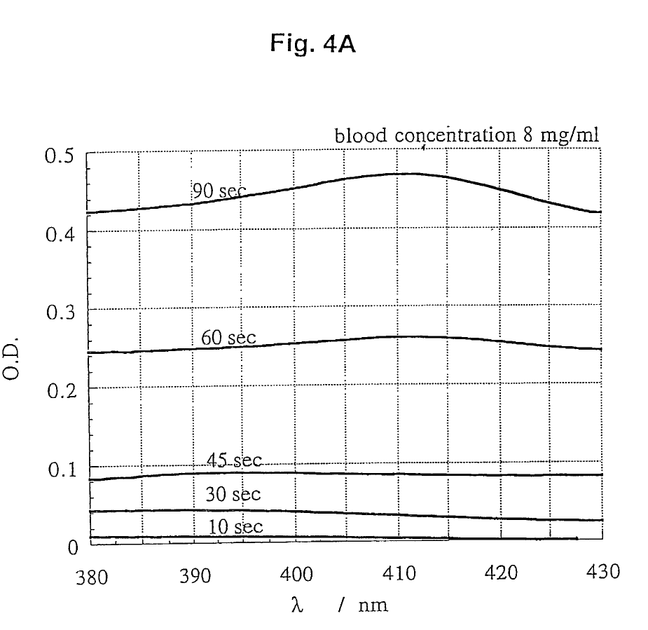

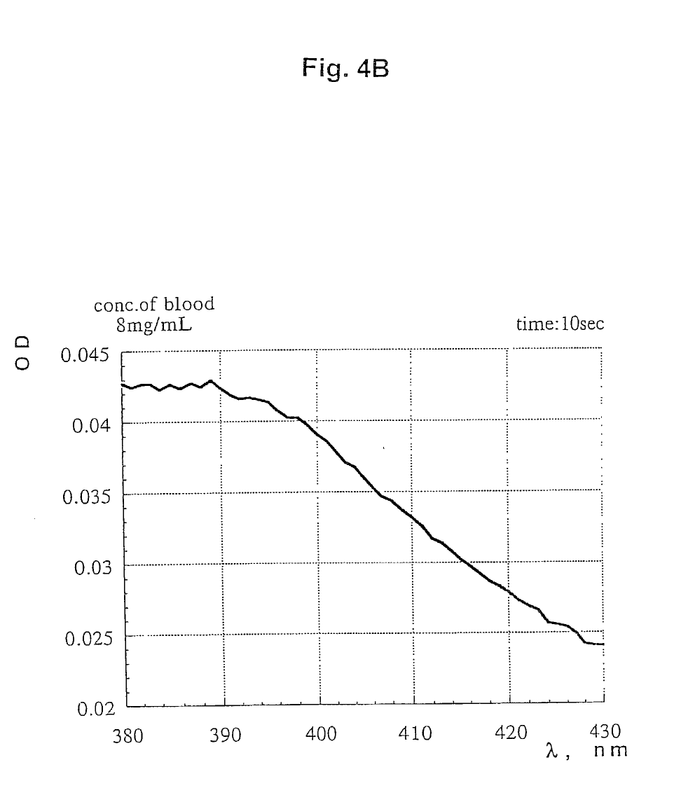

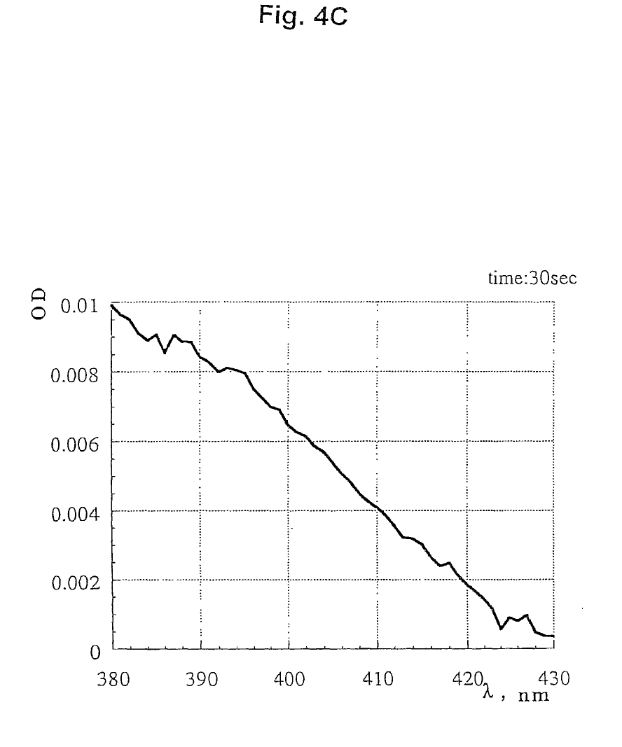

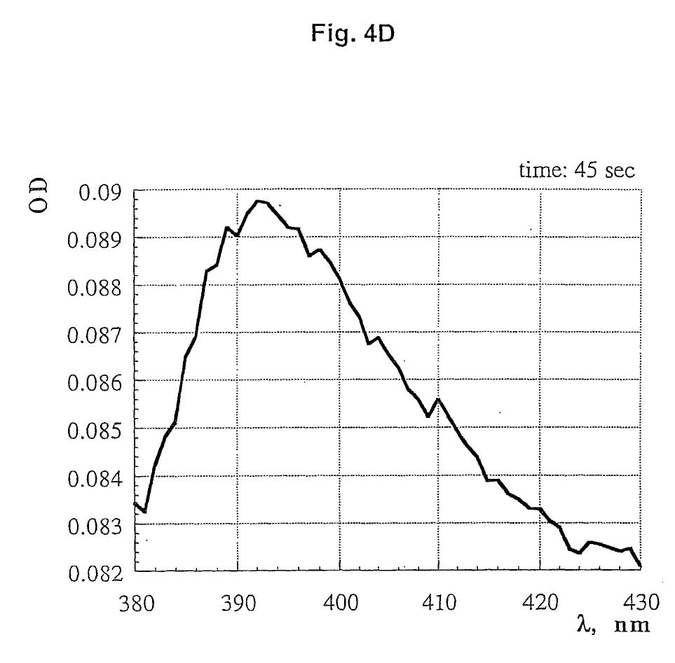

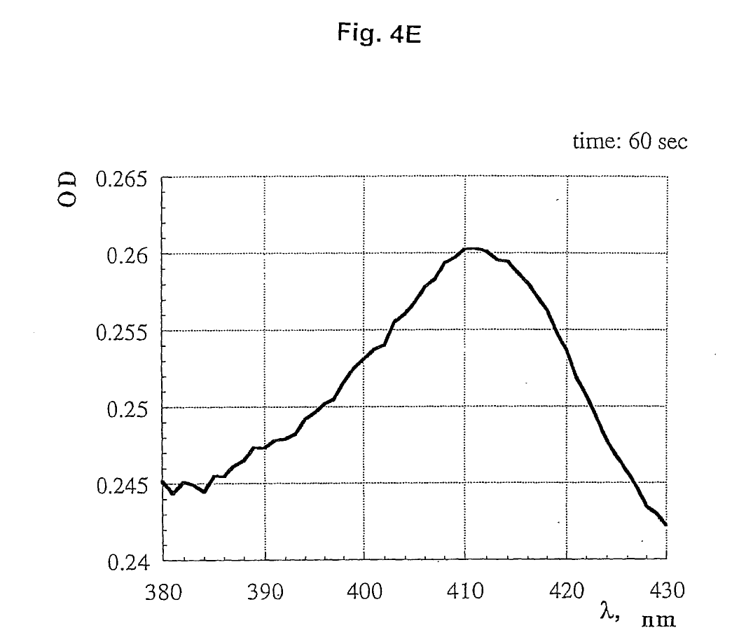

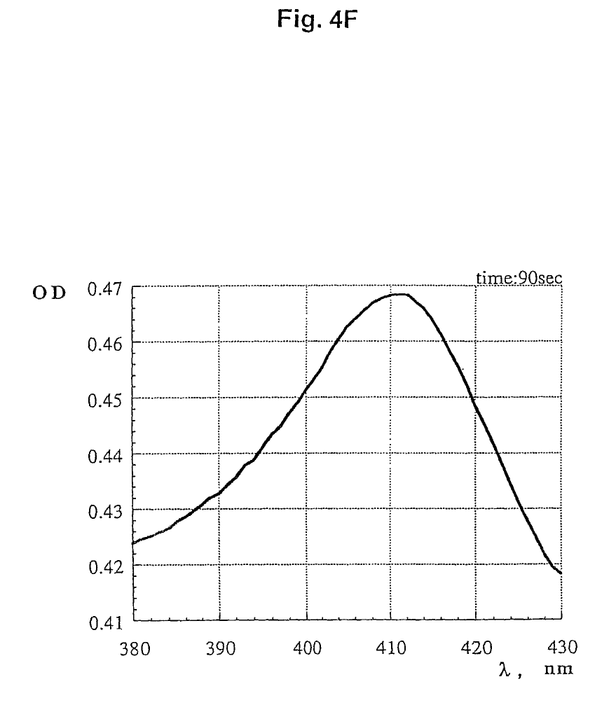

The spectra in FIGS. 3A-E and 4A-F clearly demonstrate a shift of the absorbency band at 386-390 nm and the formation of a peak at 410-412 nm, the latter being typical of hemoglobin.

The results demonstrate that PAA forms a coating on Isoplast® that induces detectable blood coagulation within a period of 30-150 seconds, even for blood concentrations as low as 2.5 mg/ml.

It will be appreciated by persons skilled in the art that the present invention is not limited by what has been particularly shown and described herein above. Rather the scope of the invention is defined by the claims which follow.

1. A device for in vitro detection of blood comprising a support having immobilized thereon at least one poly electrolyte reactant capable of reacting with blood, said reaction resulting in an optical change.

2. A device according to claim 1 wherein the support is made of glass or plastic.

3. A device according to claim 2 wherein the support is made of a poly urithane.

4. A device according to claim 1 wherein the poly electrolyte reactant is selected from the group consisting of poly acrylic acid, poly aspartic acid, poly glutamic acid and cellulose acetic acid.

5. A device according to claim 1 wherein the support is made of isoplast and the poly electrolyte reactant is poly acrylic acid.

6. A system for in vitro detection of blood comprising
a support having immobilized thereon at least one poly electrolyte reactant capable of reacting with blood, said reaction resulting in an optical change; and
a detection unit in communication with the support for detecting the optical change.

7. A system according to claim 6 wherein the support is made of glass or plastic.

8. A system according to claim 7 wherein the support is made of a poly urithane.

9. A system according to claim 6 wherein the poly electrolyte reactant is selected from the group consisting of poly acrylic acid, poly aspartic acid, poly glutamic acid and cellulose acetic acid.

10. A system according to claim 6 wherein the detection unit is a spectrophotometer.

11. A system according to claim 6 wherein the support is made of isoplast and the poly electrolyte reactant is poly acrylic acid.

12. A method for determining the presence and/or concentration of blood in a sample comprising the steps of
introducing the sample to a support, the support having immobilized thereon at least one poly electrolyte reactant capable of reacting with blood, said reaction resulting in an optical change; and
receiving optical data from the support.

13. A method according to claim 12 wherein the optical data is received by a detection unit.

14. A method according to claim 12 wherein the support is made of glass or plastic.

15. A method according to claim 14 wherein the support is made of a poly urithane.

16. A method according to claim 12 wherein the poly electrolyte reactant is selected from the group consisting of poly acrylic acid, poly aspartic acid, poly glutamic acid and cellulose acetic acid.

17. A method according to claim 12 further comprising the step of analyzing the optical data received from the support.

18. A diagnostic kit for determining the presence and/or concentration of blood in a sample comprising the device according to claim 1.

19. A diagnostic kit for determining the presence and/or concentration of blood in a sample comprising the system according to claim 6.
